(19) United States
(12) Patent Application Publication
(10) Pub. No.: US 2007/0179419 A1
(43) Pub. Date: Aug. 2, 2007

(54) LOW FRICTION COATINGS FOR ADHESIVE DRESSINGS AND METHOD OF MANUFACTURE THEREOF

(75) Inventor: Scott S. Simpson, Woodstock, CT (US)

Correspondence Address: CANTOR COLBURN, LLP, 55 GRIFFIN ROAD SOUTH, BLOOMFIELD, CT, 06002, US

(73) Assignee: WORLD PROPERTIES, INC., Lincolnwood, IL (US)

(21) Appl. No.: 11/668,259

(22) Filed: Jan. 29, 2007

Related U.S. Application Data
(60) Provisional application No. 60/762,806, filed on Jan. 27, 2006.

(51) Int. Cl. A61F 15/00

(52) U.S. Cl. 602/57

(57) ABSTRACT

An adhesive dressing comprising a conformable, elastomeric backing layer comprising a first side and a second side, a pressure-sensitive adhesive disposed on the first side, and a topcoat applied to the second side, wherein the topcoat comprises a friction-reducing surface finish, wherein the coefficient of function of the topcoat is equal to or less than 0.6 as measured by ASTM 1894-95.

### CROSS-REFERENCE TO RELATED APPLICATION

This application claims priority to U.S. Provisional Application Ser. No. 60/762,806 filed Jan. 27, 2006, which is incorporated by reference herein in its entirety.

### BACKGROUND

The present invention relates to adhesive dressings, and, more particularly, to dressings having low friction coatings thereon, as well as methods of manufacture of the dressings.

Adhesive dressings generally comprise a backing layer with a pressure-sensitive adhesive disposed on one side of the backing layer for adhering to skin. The opposite side of the adhesive dressing is non-adhesively coated and typically exposed to the environment. Adhesive dressings for use in wound care applications (where the dermis has been cut, scraped, and the like) generally have a treatment layer disposed on the adhesive-coated side of the backing layer. The treatment layer can be an absorbent pad capable of absorbing liquids expressed by the wound, or in applications wherein wounds express an increased volume of liquids, such as in cases of burns and pressure sores, the treatment layer can be a super-absorbent absorbent pad incorporating hydrogels and/or hydrocolloids. The treatment layer can also provide for drug or medicament delivery to a breached or non-breached dermis. Adhesive dressings without a treatment layer are also used, for example for post-operative wound closure, sterile field maintenance, I.V. securement, and the like.

A wide variety of materials have been used as backing layers in adhesive dressings, including smooth, flexible plastics such as polyethylene, polyvinyl chloride, and polyvinylidene-chloride. However, elastomers such as polyurethane, ethylene propylene-diene-monomer (EPDM), and polybutadiene are perceived to yield a softer, more flexible dressing compared to those produced from smooth plastic backings. The backing layer can also be embossed with patterns (e.g., dimples) and/or surface finishes (e.g., a matte finish) to mimic the look and feel of cloth, and also to make the dressing less noticeable when applied to skin. Although elastomers comprising these features have proven to be commercially successful, these materials generally exhibit high friction when in use, especially when in contact with other compliant materials such as skin, fabrics, plastics, and so forth. In these situations, the edges of the dressing frequently lift from the skin, fold over, and/or roll, which further proliferates the detachment of the dressing and causes discomfort to the user. In light of the foregoing, a need in the art exists for elastomeric adhesive dressings having an outer surface having a lower coefficient of friction.

### SUMMARY

The above-described drawbacks and disadvantages are alleviated by an adhesive dressing comprising a conformable, elastomeric backing layer comprising a first side and an opposite second side, a pressure-sensitive adhesive disposed on the first side, and a topcoat applied to the second side, wherein the topcoat comprises a friction-reducing surface finish, and wherein the coefficient of friction of the topcoat is equal to or less than 0.6 as measured by ASTM 1894-95.

In a second embodiment, a method of making an adhesive dressing is disclosed. The method comprises, forming a backing layer, disposing a topcoat having an inner surface and an outer surface onto a first side of the backing layer, wherein the inner surface is disposed on the backing layer, and wherein the outer surface comprises a friction-reducing surface finish having a coefficient of friction equal to or less than about 0.6 as measured by ASTM 1894-95.

In a third embodiment, a method of making an adhesive dressing is disclosed. The method comprises, disposing a curable topcoat composition having an inner surface and an outer surface onto a first side of a backing layer, wherein the inner surface is disposed on the first side of the backing layer, texturing at least a portion of the outer surface of the topcoat composition, and curing the curable topcoat composition to provide at least a portion of the outer surface with a friction-reducing surface finish having a coefficient of friction equal to or less than about 0.6 as measured by ASTM 1894-95.

In a fourth embodiment, a method of making an adhesive dressing is disclosed. The method comprises, disposing a topcoat composition having an inner surface and an outer surface onto a first side of a backing layer, wherein the inner surface is disposed on the first side of the backing layer, and texturing at least a portion of the outer surface of the topcoat composition to provide at least a portion of the outer surface with a friction-reducing surface finish having a coefficient of friction equal to or less than about 0.6 as measured by ASTM 1894-95.

The above-described and other features will be appreciated and understood by those skilled in art from following detailed description, drawings, and appended claims.

### DESCRIPTION OF THE DRAWINGS

FIG. 1 is a side view of an exemplary adhesive dressing.

FIG. 2 is a partial and detailed view of an exemplary topcoat.

FIG. 3 is a side view of an exemplary coating line.

FIG. 4 a side view of an exemplary production line.

### DETAILED DESCRIPTION

The inventors hereof have developed a coating that can be applied to the backing layers of adhesive dressings that reduces the coefficient of friction (COF) of the backing layer, thereby reducing the occurrence of edge-lift and dressing detachment. In a particularly advantageous feature, the coating provides a matte finish that further decreases the coatings coefficient of friction, makes the dressing less visible when worn, and produces a more natural and more visually appealing appearance. In addition, the presence of a low COF coating allows the use of a weaker (less tacky) adhesive, because the dressing is less likely to be inadvertently removed when rubbed against clothing, bedding, and so forth. This in turn reduces the force needed to remove the dressing and decreases user discomfort during the dressing removal.

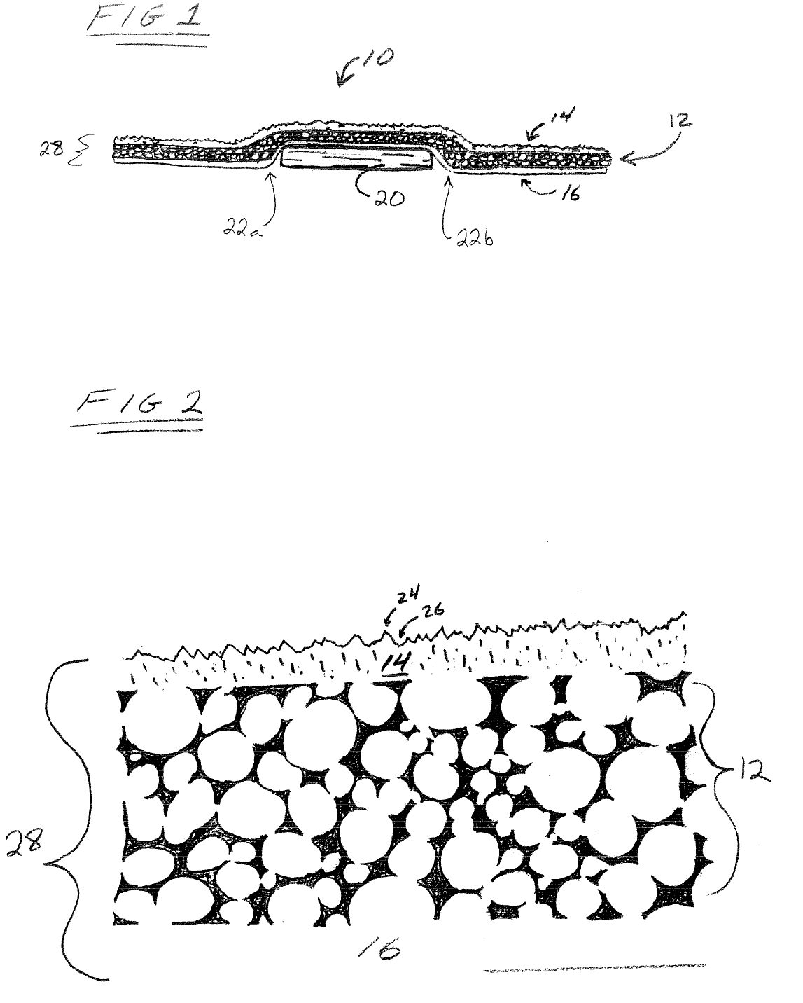

Referring now to FIG. 1, a side view of an exemplary adhesive dressing, generally designated 10, is disclosed. In the illustration, the adhesive dressing 10 comprises a film laminate 28 and a treatment layer 20. The film laminate 28 comprises a backing layer 12 coated on one side with a topcoat 14 and on the opposite side with a pressure sensitive adhesive layer 16. As shown, the treatment layer 20 (e.g., an absorbent pad) is adhered to the adhesive layer 16. It is to be understood that adhesive dressings without a treatment layer 20, or layers, are also within the scope of the present invention.

When present, the treatment layer 20 is disposed on the adhesive layer 16, usually medially positioned between the transverse edges 22a, 22b as shown. As illustrated in FIG. 1, the treatment layer 20 comprises an absorbent pad, although it is to be understood that the treatment layer 20 can comprise any treatment device that is intended to be placed over an area of the skin. For example, the treatment layer 20 can be any of the layers conventionally used for absorbing wound fluids (e.g., serum or blood) as known in the wound healing art, including foams (e.g., hydrophilic polyurethane foams, sponges, gauzes, woven fabrics, nonwoven fabrics), or a carded web of viscose staple fibers. Further, superabsorbents such as hydrocolloids or hydrogels can be dispersed in the treatment layer 20 to improve liquid absorbency and retention. In other embodiments, a hydrogel, a drug release layer, a hydration source, or the like, can be employed as the treatment layer 20.

The uncompressed thickness and area of the treatment layer 20 will depend on the intended use of the adhesive dressing. The uncompressed thickness can be, for example, about 0.5 millimeters (mm) to about 10 mm, in particular about 1 mm to about 4 mm, and the area is typically about 1 square centimeter (cm²) to about 200 cm², more specifically about 4 cm² to about 100 cm².

The treatment layer 20 can further comprise an additional, outer layer disposed on the skin-contacting surface of the treatment layer 20 (not shown). The outer layer allows fluid to pass from the skin (e.g., a wound site) through the outer layer, but blocks or restricts flow of the fluid back through the outer layer onto the skin (or wound). Such non-wetting outer layers can be made from porous, non-woven fabrics comprising a layer of hydrophobic fibers, or having a hydrophobic finish applied to at least the outer surface thereof. The outer layer can also be formed from a thermoplastic film-forming polymer. Preferably, the polymer is conformable but not substantially elastomeric. Suitable polymers include, but are not limited to, polyethylene, polypropylene, polyester, polyamides such as nylons, fluoropolymers such as polyvinylidene-fluoride (PVDF) or polytetrafluoroethylene (PTFE), and mixtures thereof. In one specific embodiment, the outer layer is preferably a polyolefin film, having, for example, a thickness of about 10 to about 200 micrometers, more specifically about 25 to about 100 micrometers. When the dressing is used as a wound dressing, the outer layer is advantageously hydrophobic, so as to reduce adherency of the top sheet to the wound.

The backing layer 12 is preferably substantially semi-impermeable, that is, permeable to water vapor but not permeable to liquids such as water or wound exudate. Preferably, the backing layer 12 is also microorganism-impermeable. A suitable backing layer 12 can have a moisture vapor transmission rate (MVTR) of, for example, equal to or greater than 500 grams of water per square meter per 24 hrs (g/m²/day), or more specifically, equal to or greater than 1000 g/m²/day, or even more specifically equal to or greater than 1500 g/m²/day, as determined by ASTM E96 at 100° F. and 50% relative humidity. The thickness of the backing layer 12 will vary depending on the materials employed, the desired physical properties (e.g., tensile strength, tear resistance), and intended use of the dressing. A suitable thickness is about 10 to about 1000 micrometers, more specifically about 100 to about 500 micrometers.

The backing layer 12 is formed from a conformable elastomer. Suitable polymers for forming the backing layer 12 include elastomeric copolyester ethers, polyurethanes, blends of polyurethanes and polyesters, silicones, and polyalkoxyalkyl acrylates and methacrylates. As shown in FIG. 1, the backing layer 12 is formed from high-density polyurethane foam that predominantly comprises an open-cell structure. A suitable backing layer 12 is the polyurethane film available under the registered trademark PORON, from Rogers Corp., Rogers, Conn.

The backing layer 12 is coated on one side with a pressure-sensitive adhesive layer 16. The adhesive layer 16 can be, for example, based on acrylate ester copolymers, polyvinyl ethyl ether, polyurethane, or the like. Suitable adhesives include copolymers of 2-ethylhexyl acrylate and vinyl acetate in ratios of approximately 60 to 70 parts of the acrylate and 30 to 40 parts of the vinyl acetate. The adhesive copolymers may also comprise small amounts of N-tertiary butylacrylamide as a third monomer and a cross-linking agent. A specific adhesive is a copolymer of approximately 70% 2-ethylhexyl acrylate and 30% vinyl acetate comprising from 0.01 to 1% of a silane cross-linking agent. Water-based adhesives and hot melt adhesives can also be used. The adhesive is deposited on the backing layer 12 by solvent spreading, transfer coating, extrusion, or other known methods. The adhesive layer 16 can be about 1 to about 100 micrometers in thickness, or more specifically about 5 to about 50 micrometers in thickness, or even more specifically about 10 to about 25 micrometers in thickness. In addition, although not illustrated, the adhesive layer 16 can comprise multiple layers comprising various adhesives.

As is conventional, a pair of removable release sheets (not shown) can be provided to protect the adhesive face and optional treatment layer 20 during storage and prior to application to the skin of a user, for example, the wound of a patient.

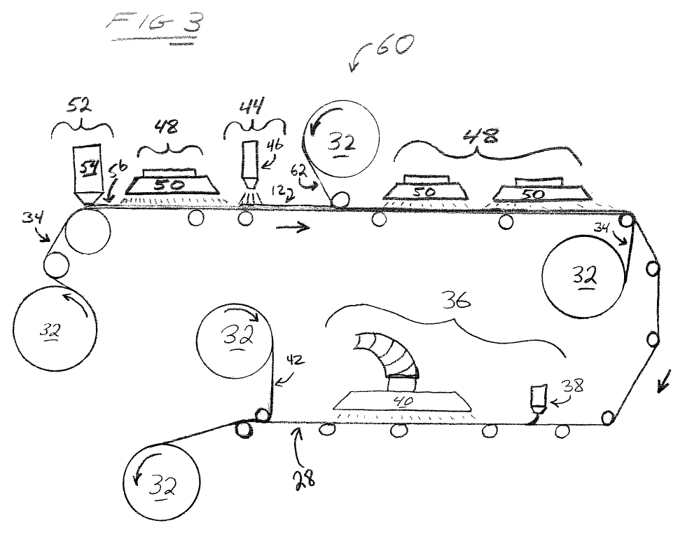

Referring now to FIG. 2, an exemplary partial and detailed view of the film laminate 28 is illustrated. In the illustration, a topcoat 14 is disposed on a backing layer 12, onto which an adhesive layer 16 is adhered thereto.

The topcoat 14 preferable comprises a material that does not significantly inhibit the moisture vapor transmission rate through the adhesive dressing 10. In addition, the topcoat 14 should be configured as to comprise a thickness that does not hinder a desired MVTR. In one embodiment, for example, the topcoat 14 can comprise a continuous layer having a thickness about 1 to about 250 micrometers. However, the thickness of topcoat 14 is desirable thinner, comprising a thickness of about 1 to about 50 micrometers, or more specifically about 1 to about 25 micrometers. In an alternative embodiment, the topcoat 14 can comprise a discontinuous layer of similar thickness, so that the MVTR is not hindered.

The topcoat 14 also reduces the coefficient of friction of the backing layer 12. More specifically, the outer surface of the topcoat 14 (i.e., the surface in contact with the environment) can comprise a dynamic coefficient of friction equal to or below 0.6, or more specifically equal to or below about 0.5, yet more specifically equal to or below about 0.4, as measured by ASTM 1894-95. A reduced coefficient of friction can be achieved using a number of methods, for example; manufacturing the topcoat 14 from a polymer comprising a lower coefficient of friction than the backing layer 12, incorporating friction-reducing media into the topcoat 14, or imparting a friction-reducing surface texture into the topcoat 14. Combinations comprising at least one of the foregoing approaches can be used.

In one embodiment, the topcoat 14 is manufactured from a polymer with an inherently low COF, and with good adhesion to the backing layer 12. While it is possible to use an adhesive between the topcoat 14 and the backing layer 12, manufacture is simplified (and cost is lower) and MVTR is maximized when a separate adhesive layer is not used. Adhesion between the polymer of the topcoat 14 and the backing layer 12 is preferably stronger than that between the pressure sensitive adhesive layer 15 and the skin of the user. Exemplary polymers include various curable epoxy, (meth)acrylate and siloxane polymers, that cure utilizing either thermal or photosensitive initiators. (Meth)acrylate polymers are derived from cure of compositions comprising multifunctional (meth)acrylates, i.e., molecules comprising at least two (meth)acrylate functional groups of formula (I)

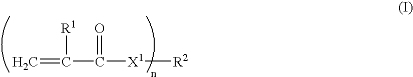

wherein R¹ is hydrogen or methyl; X¹ is O or S; R² is substituted or unsubstituted C₁₋₃₀ alkyl, aryl, alkylaryl, arylalkyl, or heteroaryl; and n is 2, 3, or 4. The substitution on R² includes, but is not limited to, fluorine, chlorine, bromine, iodine, C₁₋₆ alkyl, C₁₋₃ perhalogenated allyl, hydroxy, C₁₋₆ ketone, C₁₋₆ ester, N,N—(C₁₋₃) alkyl substituted amide, or a combination comprising at least one of the forgoing substituents. Preferred R² groups can include such groups as alkylene and hydroxy-alkylene disubstituted bisphenol-A or bisphenol-F ethers, especially the brominated forms of bisphenol-A and bisphenol-F.

Suitable polymerization initiators for (meth)acrylates include photoinitiators that promote polymerization of the components upon exposure to ultraviolet radiation, for example phosphine oxide photoinitiators such as the IRGACURE® and DAROCUR™ series of phosphine oxide photoinitiators available from Ciba Specialty Chemicals; the LUCIRIN® series from BASF Corp.; and the ESACURE® series of photoinitiators. Other useful photoinitiators include ketone-based photoinitiators, such as hydroxy- and alkoxyalkyl phenyl ketones, and thioalkylphenyl morpholinoalkyl ketones. Also suitable are benzoin ether photoinitiators. Thermal initiators, such as various peroxides, can also be used. The polymerization initiator may be used in an amount of about 0.01 to about 10 weight percent, specifically about 0.1 to about 0.5 weight percent, based on the total weight of the curable composition.

Suitable siloxane polymers are derived from the reaction of an organopolysiloxanes having at least two alkenyl groups per molecule with an organopolysiloxanes having at least two silicon-bonded hydrogen atoms per molecule. Alkenyl-substituted organopolysiloxanes are generally represented by the formula:

MₐD_{b}T_{c}Q_{d},

wherein subscripts a, b, c, and d are zero or a positive integer, subject to the limitation that if subscripts a and b are both equal to zero, subscript c is greater than or equal to two; M has the formula R₃SiO_{1/2}; D has the formula R₂SiO_{2/2}; T has formula RSiO_{3/2}; and Q has formula SiO_{4/2}, wherein each R group independently represents hydrogen, alkenyl groups, substituted and unsubstituted monovalent hydrocarbon groups having from one to forty, preferably one to six carbon atoms each, subject to limitation that at least two of the R groups are alkenyl R groups. Suitable alkenyl R groups are exemplified by vinyl, allyl, butenyl, pentenyl, hexenyl, and heptenyl, with vinyl being particularly preferred. The alkenyl group can be bonded at molecular chain terminals, in pendant positions on the molecular chain, or both. Non-alkenyl R groups are exemplified by substituted and unsubstituted alkyl groups such as methyl, ethyl, propyl, butyl, pentyl, and hexyl; aryl groups such as phenyl, tolyl, and xylyl; aralkyl groups such as benzyl and phenethyl; and halogenated alkyl groups such as 3-chloropropyl and 3,3,3-trifluoropropyl. Methyl and phenyl are specifically preferred.

The alkenyl-containing organopolysiloxane can have straight chain, partially branched straight chain, branched-chain, or network molecular structure, or may be a mixture of such structures. The preferred alkenyl-containing organopolysiloxane is exemplified by trimethylsiloxy-endblocked dimethylsiloxane-methylvinylsiloxane copolymers; trimethylsiloxy-endblocked methylvinylsiloxane-methylphenylsiloxane copolymers; trimethylsiloxy-endblocked dimethylsiloxane-methylvinylsiloxane-methylphenylsiloxane copolymers; dimethylvinylsiloxy-endblocked dimethylpolysiloxanes; dimethylvinylsiloxy-endblocked methylvinylpolysiloxanes; dimethylvinylsiloxy-endblocked methylvinylphenylsiloxanes; dimethylvinylsiloxy-endblocked dimethylvinylsiloxane-methylvinylsiloxane copolymers; dimethylvinylsiloxy-endblocked dimethylsiloxane-methylphenylsiloxane copolymers; dimethylvinylsiloxy-endblocked dimethylsiloxane-diphenylsiloxane copolymers; and mixtures comprising at least one of the foregoing organopolysiloxanes.

A suitable organopolysiloxane having at least two silicon-bonded hydrogen atoms per molecule is generally represented by the formula:

M′ₐD′_{b}T′_{c}Q′_{d},

wherein subscripts a, b, c, and d are zero or a positive integer, subject to the limitation that if subscripts a and b are both equal to zero, subscript c is greater than or equal to two; M′ has the formula R′₃SiO_{1/2}; D′ has the formula R′₂SiO_{2/2}; T′ has the formula R′SiO_{3/2}; and Q′ has the formula SiO_{4/2}, wherein each R′ group independently represents hydrogen, substituted and unsubstituted monovalent hydrocarbon groups having from one to forty, preferably one to six carbon atoms each, subject to the limitation that at least two of R groups are hydrogen. Preferably, each of the R′ groups of the organopolysiloxane having at least two silicon-bonded hydrogen atoms per molecule are independently selected from hydrogen, methyl, ethyl, propyl, butyl, pentyl, hexyl, aryl, phenyl, tolyl, xylyl, aralkyl, benzyl, phenethyl, halogenated alkyl, 3-chloropropyl, 3,3,3-trifluoropropyl, and combinations comprising at least one of foregoing. Methyl and phenyl are specifically preferred.

The hydrogen can be bonded at molecular chain terminals, in pendant positions on molecular chain, or both. The hydrogen-containing organopolysiloxane component can have straight chain, partially branched straight chain, branched-chain, cyclic, or network molecular structure, or may be a mixture of two or more selections from organopolysiloxanes with the exemplified molecular structures.

The hydrogen-containing organopolysiloxane is exemplified by trimethylsiloxy-endblocked methylhydrogenpolysiloxanes; trimethylsiloxy-endblocked dimethylsiloxane-methylhydrogensiloxane copolymers; trimethylsiloxy-endblocked methylhydrogensiloxane-methylphenylsiloxane copolymers; trimethylsiloxy-endblocked dimethylsiloxane-methylhydrogensiloxane-methylphenylsiloxane copolymers; dimethylhydrogensiloxy-endblocked dimethylpolysiloxanes; dimethylhydrogensiloxy-endblocked methylhydrogenpolysiloxanes; dimethylhydrogensiloxy-endblocked dimethylsiloxanes-methylhydrogensiloxane copolymers; dimethylhydrogensiloxy-endblocked dimethylsiloxane-methylphenylsiloxane copolymers; and dimethylhydrogensiloxy-endblocked methylphenylpolysiloxanes.

The hydrogen-containing organopolysiloxane component is used in an amount sufficient to cure the composition, preferably in a quantity that provides from about 1.0 to about 10 silicon-bonded hydrogen atoms per alkenyl group in alkenyl-containing organopolysiloxane. When the number of silicon-bonded hydrogen atoms per alkenyl group exceeds 10, foam may be produced during cure and the heat resistance of the resulting cured silicone may progressively decline.

The curable composition further comprises, generally as a component of the part containing organopolysiloxane having at least two alkenyl groups per molecule, a hydrosilylation-reaction catalyst. Effective catalysts promote the addition of silicon-bonded hydrogen onto alkenyl multiple bonds to accelerate cure. Such catalysts can include a noble metal, such as, for example, platinum, rhodium, palladium, ruthenium, iridium, or a combination comprising at least one of the foregoing. The catalyst can also include a support material, preferably activated carbon, aluminum oxide, silicon dioxide, thermoplastic resin, and combinations comprising at least one of the foregoing. Platinum and platinum compounds known for their use as hydrosilylation-reaction catalysts are preferred, and include, for example platinum black, platinum-on-alumina powder, platinum-on-silica powder, platinum-on-carbon powder, chloroplatinic acid, alcohol solutions of chloroplatinic acid platinum-olefin complexes, platinum-alkenylsiloxane complexes and catalysts afforded by microparticulation or dispersion of a platinum addition-reaction catalyst, as described above, in a thermoplastic resin such as methyl methacrylate, polycarbonate, polystyrene, silicone, and the like. Mixtures of catalysts may also be used. A quantity of catalyst effective to cure the composition is used, generally from about 0.1 to about 1,000 parts per million by weight (ppm) of metal (e.g., platinum) based on combined amounts of reactive organopolysiloxane components.

Other additives known in the art may be present in the composition used to form the topcoat, for example reinforcing fillers, ultraviolet (UV) stabilizers, pigments, dyes, antioxidants, thermal stabilizers, anti-static agents, surfactants, and the like, and a combination comprising at least one of the foregoing additives, so long as they do not significantly deleteriously affect the polymerization of the composition or the other desirable properties of the topcoat.

When a friction-reducing media is incorporated into the topcoat 14, the media can be in the form of particles, such as powders, flakes, spheres, agglomerates, fibers, fluids, and so forth, as well as combinations comprising at least one of the foregoing. Particle size will depend on the particular media used, the thickness of the topcoat, commercial availability of the media, and like considerations. Suitable media can have, for example, a largest average dimension of about 1 nanometer to about 100 micrometers, more specifically about 0.5 micrometer to about 50 micrometers. Suitable friction-reducing media include polymer particles (e.g., polytetrafluoroethylene, ultrahigh molecular weight polyethylene, siloxane), lubricants (e.g., leaching agents, silicone), mineral compounds (zinc stearate, calcium stearate, talc, silica), waxes (polyethylene, paraffin), and the like, as well as combinations comprising at least one of the foregoing. The concentration of the friction-reducing media within the topcoat 14 will depend on the specific media and the topcoat used, and can be about 1 to about 75 weight percent, based on the total weight of topcoat 14. For example, in one embodiment the topcoat 14 can comprise 15 weight percent Zonyl® polytetrafluoroethylene powder (commercially available from E. I. du Pont de Nemours and Company, Wilmington, Del.) comprising a particle size between 2.0 micrometers and 20.0 micrometers.

The friction-reducing media can be incorporated into the curable polymer composition used to form the topcoat 14 prior to application onto the backing layer 12, using conventional mixing methods such as paddle, batch, and feed mixers, and the like. The topcoat composition is then coated, cast, adhered, or otherwise applied to the backing layer.

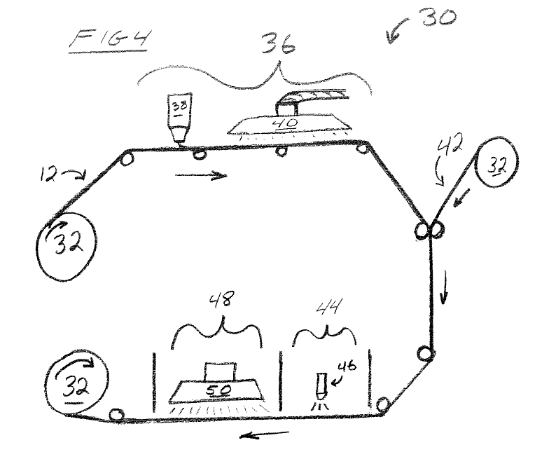

Referring now to FIG. 3, an exemplary coating production line is illustrated. In the illustration, a spool 32 supplies a coated release paper 34 to a knife-over-roll coating zone 52 wherein a knife coater 54 disperses an uncured foam 56 thereon at a desired thickness. The uncured foam 56 and the coated release paper 34 then travel through a curing zone 48, wherein the uncured foam 56 is cured via an energy source 50 (e.g., light, heat) to form a backing layer 12 on the coated release paper 34.

The backing layer 12 on the coated release paper 34 is conveyed on support rollers through a coating zone 44 (as illustrated by direction arrows), wherein a topcoat 14 is applied to the backing layer 12 via a nozzle 46. Once the topcoat 14 has been applied, an optional masking film 62 can be applied over the topcoat 14 to protect the topcoat during further processing. The topcoat 14 is then cured in a curing zone 48 comprising an energy source 50 (e.g., light, heat). Thereafter, the coated release paper 34 is stripped from the backing layer 12 and the backing layer 12/topcoat 14 travel through an adhesive application zone 36, wherein an applicator 38 applies an adhesive layer 16 onto the backing layer 12 to form a film laminate 28. Optionally, the film laminate 28 advances under a cooling apparatus 40, which removes heat from the adhesive. Once cooled below the melt temperature of the adhesive, a removable protective film 42 can be disposed on the adhesive layer 16 and the protectively coated film laminate 28 is spooled.

Referring now to FIG. 4, a side view of another exemplary coating line, generally designated 30, is illustrated. In the illustration a spool 32 supplies a backing layer 12, which is conveyed on support rollers through an adhesive application zone 36 wherein an applicator 38 applies the adhesive layer 16 onto the backing layer 12. The film advances under a cooling apparatus 40 capable of removing heat from the adhesive. Once cooled below the melt temperature of the adhesive, a protective film 42 can be adhered to the adhesive layer 16. Thereafter the film is advanced to a coating zone 44, wherein the topcoat 14 is applied to the backing layer 12 via a nozzle 46. Once the topcoat 14 has been applied, it is cured within a curing zone 48 via an energy source 50 (e.g., light, heat) and the protectively coated film laminate is spooled.

In an alternative embodiment, the topcoat 14 is manufactured to have a friction-reducing finish on the outer surface (i.e., the surface not in contact with the backing layer). As the exemplary illustration in FIG. 2 shows, the outer surface of the topcoat 14 has an irregular surface finish, having peaks 24 and valleys 26. Without being bound by theory, it is hypothesized that in use, when objects (e.g., skin, linens) contact the outer surface, the object contacts the peaks 24, which is an effectively reduced contact area compared to a smooth surface not comprising peaks 24 and valleys 26, and which reduction in contact surface area results in a reduction of the coefficient of friction of the outer surface. Thus, in one embodiment, the outer surface is provided with surface roughness (Ra) of equal to or less than about 10.0, or more specifically equal to or less than about 5.0, as measured by ASME/ANSI B46.1 (1995). Desirably, the friction-reducing surface finish also makes the article less visible during use, as a rough surface finish reflects less light than a comparable article having a smooth surface finish.

A friction-reducing finish can be provided, for example, by distributing a friction-reducing media onto the outer surface of the topcoat 14. The media can be adhered using an adhesive, but is preferably distributed onto the outer surface prior to cure of the topcoat 14. This can be achieved using a vibratory distribution apparatus or other apparatus capable of distributing the friction-reducing media across the surface of the topcoat 14 with the desired uniformity and coverage. Cure of the topcoat 14 results in adherence of the friction reducing media to the topcoat 14, with the friction-reducing media extending from the topcoat's surface, thereby reducing the coefficient of friction of the surface.

In the alternative, or in addition, a friction-reducing surface finish can be imparted by embossing (contacting the topcoat 14 with a textured surface prior to cure, followed by cure of the composition), or by texturing after cure of the topcoat composition. Texturing after cure can be by chemical means, e.g., treatment with acid or base, or by physical means, for example abrasion. The finish can be imparted to the topcoat 14 before assembly of the adhesive dressing, during assembly of the adhesive dressing, or after assembly of the adhesive dressing.

In a specific embodiment, the textured surface is imparted during manufacture of the adhesive dressing. For example, referring again to FIG. 3, after the topcoat 14 is deposited onto the backing layer 12 via applicator 46, the masking film 62 can be layered thereon, wherein the masking film 62 comprises a textured surface having a negative image of the desired topcoat 14 surface finish. Once layered thereon, the topcoat 14 is cured in curing zone 48. Once the masking film 62 is removed, the topcoat 14 will comprise the desired surface texture.

In another embodiment (not shown), a coated release paper 34 can comprise a textured surface on at least one side. The backing layer 12 can be coated onto the textured surface utilizing a knife-over-roll coating zone 52 and then cured in a curing zone 48. Thereafter, the coated release paper 34 can be removed from the backing layer 12 producing a textured backing layer, onto which a topcoat 14 can be applied and cured. The topcoat substantially conforms to the textured surface of the backing layer, such that the outer surface of the dressing has the desired texture.

An adhesive layer 16 can then be applied to the surface of the textured backing layer that is opposite the topcoat 14. The resulting film laminate 28 comprises a textured, friction reducing finish.

In yet another embodiment, again referring to FIG. 3, a non-textured masking film 62 is applied over the topcoat 14 and the masking film 62/topcoat 14 laminate can be passed through a textured nip roll (not shown), which is capable of imparting the desired surface finish to the outer surface of topcoat 14. The topcoat 14 can then be cured in curing zone 48. In a further embodiment, an embossing belt can be employed in lieu of the textured nip roll. If so, the topcoat can be cured on the embossing belt (e.g., heat, UV light), then stripped therefrom.

In yet another embodiment (not shown), the topcoat 14 can be cast onto a masking film 62 having a negative of the desired texture on a surface thereof and cured. The topcoat 14 can then be adhered to a backing layer 12.

Yet in another embodiment, referring now to FIG. 4, a backing layer 12 can comprise a friction reducing surface finish thereon, which can be imparted to the backing layer 12 as described above (e.g., embossing roll, embossing film, embossing belt, casting onto a textured release layer). The textured backing layer 12 can then be coated with a topcoat 14 in the coating zone 44 and then cured in a curing zone 48. The film laminate 28 produced will comprise a friction reducing surface when the topcoat substantially conforms to the textured surface of the backing layer.

Utilizing the various manufacturing methods discussed above, additional embodiments of the film laminate 28 can be produced. More specifically, additional layers can be incorporated into the film laminate 28 to provide enhanced properties, functionality, and the like. For example, in one embodiment a polyurethane layer can be incorporated between the backing layer 12 and the adhesive layer 16, wherein the polyurethane layer comprises a thickness of about 25 micrometers and is capable of providing a bacterial barrier. In yet another embodiment, a drug release layer can be disposed between the backing layer 12 and the adhesive layer 16, wherein the drug release layer comprises a thickness of about 50 micrometers and is capable of eluting a drug or medicament.

The adhesive dressings comprising a treatment layer 20 are useful in a variety of dermal applications, for example wound site care, transdermal drug delivery, and the like. Adhesive dressings without treatment layer 20 are also useful for maintaining sterile sites (e.g., I.V. sites, post-operative sites). The conformable, elastomeric adhesive dressings have a reduced coefficient of friction of the backing layer 12, thereby reducing the occurrence of edge-lift and dressing detachment. This advantageously decreases the frequency of dressing changes, maintains dermal treatment, and reduces discomfort for the wearer. In a particularly advantageous feature, the adhesive dressings also comprises a textured finish and a topcoat 14, which further decreases the coefficient of friction and also produce a more natural and more visually appealing appearance and less visible by providing a lower gloss level (e.g., a matte finish). In addition, the presence of a low COF coating allows for the use of a weaker (less tacky) adhesive, because the dressing is less likely to be inadvertently removed while rubbed against clothing, bedding, and so forth. This in turn reduces the force needed to remove the dressing, decreasing discomfort of the user during dressing removal, while also reducing the cost incurred by manufacturers as more expensive higher tack adhesives can be replaced with lower tack alternatives.

The following non-limiting examples were prepared, to show the improvement in the coatings of the present invention compared to a competitive material. Examples A and B were prepared in accordance with the materials and procedures described above. Gloss, COF, and MVTR were measured as set forth in the Table.

| | | COF on | MVTR - | | |
|---|---|---|---|---|---|
| | | Stainless | upright | | |
| | Gloss | Steel | ASTM E96 | | |
| | ASTM D | ASTM D | at 37° C. | MVTR - | COF on |
| | 523 | 1894 | and 0% RH | inverted | cotton |
| Example A | 2.1 | 0.2 | 1948 | 3939 | 0.4 |
| Coating | | | | | |
| Example B | 4.7 | 0.3 | 1684 | 3450 | NA |
| Coating | | | | | |
| Uncoated | 11.1 | 1.1 | 3210 | 6668 | 0.5 |
| low COF | | | | | |
| matte finish | | | | | |
| foam | | | | | |
| Competitive | 3.5 | 5.3 | 3446 | Leakage | 0.8 |
| Material | | | | | |

As the above Examples A and B show, the coatings in accordance with the invention have lower COF and MVTR values.

Unless defined otherwise, technical and scientific terms used herein have the same meaning as is commonly understood by one of skill in the art to which this invention belongs. The terms “first,” “second,” and the like, do not denote any order, quantity, or importance but rather are used to distinguish one element from another. Also, the terms “a” and “an” do not denote a limitation of quantity, but rather denote the presence of at least one of the referenced item, and the terms “front”, “back”, “bottom”, and/or “top”, unless otherwise noted, are merely used for convenience of description, and are not limited to any one position or spatial orientation. If ranges are disclosed, the endpoints of all ranges directed to the same component or property are inclusive and independently combinable. The modifier “about” used in connection with a quantity is inclusive of the stated value and has the meaning dictated by the context (e.g., includes the degree of error associated with measurement of the particular quantity). The term “(meth)acrylate” refers to “methacrylate” and/or “acrylate”.

Although the Figures show a particular form of adhesive dressing, the present invention can be implemented on any suitable dressing such as an adhesive strip bandage or surgical dressing of any shape or size. Accordingly, the present invention has been described with reference to specific embodiments. Other embodiments of the present invention will be apparent to one of ordinary skill in the art. It is, therefore, intended that the claims set forth below not be limited to the embodiments described above.

What is claimed is:

1. An adhesive dressing, comprising
a conformable, elastomeric backing layer comprising a first side and a second side opposite the first side;
a pressure-sensitive adhesive layer disposed on the first side of the backing layer; and,
a topcoat having an inner surface and an outer surface, wherein the inner surface is disposed on the second side of the backing layer and wherein the outer surface comprises a friction-reducing surface finish having a coefficient of friction equal to or less than about 0.6 as measured by ASTM 1894-95.

2. The adhesive dressing of claim 1, wherein the friction-reducing surface finish has a coefficient of friction equal to or less than about 0.5 as measured by ASTM 1894-95.

3. The adhesive dressing of claim 1, wherein the friction-reducing surface finish has a coefficient of friction equal to or less than about 0.4 as measured by ASTM 1894-95.

4. The adhesive dressing of claim 1, wherein the friction-reducing surface finish is a matte finish.

5. The adhesive dressing of claim 1, wherein the friction-reducing surface finish has an average surface roughness of approximately equal to or less than about 5.0 as measured by ASME/ANSI B46.1 (1995).

6. The adhesive dressing of claim 1, wherein the backing layer is in the form of a foam.

7. The adhesive dressing of claim 6, wherein the foam comprises a polyurethane or a siloxane polymer.

8. The adhesive dressing of claim 1, wherein the backing layer has a moisture vapor transmission rate equal to or greater than 500 grams water per square meter per 24 hrs as determined by ASTM E96 at 100° F. and 90 relative % humidity.

9. The adhesive dressing of claim 1, wherein the topcoat comprises a (meth)acrylate, silicone, or epoxy polymer.

10. The adhesive dressing of claim 13, wherein the outer surface of the topcoat is textured.

11. The adhesive dressing of claim 1, wherein the second side of the backing layer is textured.

12. The adhesive dressing of claim 1, wherein the second side of the backing layer is textured, and further wherein the topcoat substantially conforms to the textured surface.

13. The adhesive dressing of claim 1, further comprising a treatment layer disposed on a side of the pressure-sensitive adhesive layer opposite the backing layer.

14. The adhesive dressing of claim 13, wherein the treatment layer comprises an absorbent pad.

15. The adhesive dressing of claim 13, wherein the treatment layer comprises a drug release layer.

16. The adhesive dressing of claim 1, further comprising friction-reducing media disposed within or on the topcoat.

17. A method of making an adhesive dressing, comprising
forming a backing layer;
disposing a topcoat having an inner surface and an outer surface onto a first side of the backing layer, wherein the inner surface is disposed on the backing layer; and,
wherein the outer surface comprises a friction-reducing surface finish having a coefficient of friction equal to or less than about 0.6 as measured by ASTM 1894-95.

18. The method of claim 17, further comprising disposing a pressure-sensitive adhesive layer onto a side of the backing layer opposite the first side.

19. The method of claim 17 wherein the outer surface has a matte finish.

20. A method of making an adhesive dressing, comprising
disposing a curable topcoat composition having an inner surface and an outer surface onto a first side of a backing layer, wherein the inner surface is disposed on the first side of the backing layer;
texturing at least a portion of the outer surface of the topcoat composition; and
curing the curable topcoat composition, to provide at least a portion of the outer surface with a friction-reducing surface finish having a coefficient of friction equal to or less than about 0.6 as measured by ASTM 1894-95.

21. The method of claim 20, wherein texturing comprises contacting the outer surface with a negative of the desired texture.

22. The method of claim 21, wherein the negative is disposed on a nip roller, embossing belt, masking film, coated release paper, or a combination comprising at least one of the foregoing.

23. The method of claim 20, wherein texturing is by distributing friction reducing media on the outer surface.

24. The method of claim 20, further comprising disposing a pressure-sensitive adhesive layer onto a side of the backing layer opposite the first side.

25. A method of making an adhesive dressing, comprising
disposing a topcoat composition having an inner surface and an outer surface onto a first side of a backing layer, wherein the inner surface is disposed on the first side of the backing layer;
texturing at least a portion of the outer surface of the topcoat composition to provide at least a portion of the outer surface with a friction-reducing surface finish having a coefficient of friction equal to or less than about 0.6 as measured by ASTM 1894-95.

26. The method of claim 25, further comprising disposing a pressure-sensitive adhesive layer onto a side of the backing layer opposite the first side.

27. A method of making an adhesive dressing, comprising
disposing a topcoat composition having an inner surface and an outer surface onto a first side of a backing layer, wherein the inner surface is disposed on the first side of the backing layer, wherein the first side of the backing layer is textured, and wherein at least a portion of the outer surface of the topcoat composition has a friction-reducing surface finish having a coefficient of friction equal to or less than about 0.6 as measured by ASTM 1894-95.

28. The method of claim 27, wherein the first side of the backing layer is textured by casting a backing layer composition onto a negative of the textured surface.

29. The method of claim 28, wherein the negative of the textured surface is on a release layer.
